(19) United States
(12) Patent Application Publication
(10) Pub. No.: US 2017/0367610 A1
(43) Pub. Date: Dec. 28, 2017

(54) METHOD AND APPARATUS OF MONITORING A PATIENT FOR MOTOR MANIFESTATIONS RELATED TO SEIZURE ACTIVITY

(71) Applicant: Brain Sentinel, Inc., San Antonio, TX (US)

(72) Inventor: Michael R. Girouard, Shavano Park, TX (US)

(73) Assignee: Brain Sentinel, Inc., San Antonio, TX (US)

(21) Appl. No.: 15/539,581

(22) PCT Filed: Dec. 23, 2015
(86) PCT No.: PCT/US2015/000475

§ 371 Date: Jun. 23, 2017

Related U.S. Application Data
(63) Continuation-in-part of application No. 14/920,665, filed on Oct. 22, 2015.
(60) Provisional application No. 62/149,434, filed on Apr. 17, 2015.
(60) Provisional application No. 62/096,331, filed on Dec. 23, 2014.

(51) Int. Cl. A61B 5/0488; A61B 5/00

(52) U.S. Cl. CPC A61B 5/0488; A61B 5/4094; A61B 5/7282

(57) ABSTRACT

Methods and apparatuses are described for qualifying peaks included among a collected EMG signal and include methods where various test groupings of peaks may be constructed from among a group of peaks. Methods and apparatuses herein may facilitate identification of patterns of abnormal motor manifestation, initiation of alarms if those patterns are identified, and the organization of data for inclusion in a searchable medical database.

### CROSS-REFERENCE TO RELATED APPLICATION

This application claims priority to U.S. Provisional Patent Application No. 62/096,331 filed Dec. 23, 2014 and U.S. Provisional Patent Application No. 62/149,434 filed Apr. 17, 2015. The application also claims priority to U.S. application Ser. No. 14/920,665 filed Oct. 22, 2015. The disclosures of each of the, aforementioned applications are herein fully incorporated by reference.

### BACKGROUND

A seizure may be characterized as abnormal or excessive synchronous activity in the brain. At the beginning of a seizure, neurons in the brain may begin to fire at a particular location. As the seizure progresses, this firing of neurons may spread across the brain, and in some cases, many areas of the brain may become engulfed in this activity. Seizure activity in the brain may cause the brain to send electrical signals through the peripheral nervous system activating different muscles of the body.

Techniques designed for studying and monitoring seizures have typically relied upon electroencephalography (EEG), which characterizes electrical signals using electrodes attached to the scalp or head region of a seizure-prone individual or seizure patient. In EEG, electrodes may be positioned so as to measure such activity; that is, electrical activity originating from neuronal tissue. Alternatively, electromyography (EMG) may be used for seizure detection. In EMG, an electrode may be placed on or near the skin, over a muscle, to detect electrical activity resulting from muscle fiber activation.

Detecting an epileptic seizure using EEG typically requires attaching many electrodes and associated wires to the head and using amplifiers to monitor brainwave activity. The multiple EEG electrodes may be very cumbersome and generally require some technical expertise to apply and monitor. Furthermore, confirming a seizure may require observation in an environment provided with video monitors and video recording equipment. Unless used in a staffed clinical environment, such equipment may not be intended to determine if a seizure is in progress, but rather provide a historical record of the seizure after the incident. Such equipment is usually meant for hospital-like environments where a video camera recording or caregiver's observation may provide corroboration of the seizure, and is typically used as part of a more intensive care regimen such as a hospital stay for patients who experience multiple seizures. Upon discharge from the hospital, a patient may be sent home, often with little further monitoring.

Ambulatory devices for diagnosis of seizures are generally EEG-based, but because of the above shortcomings those devices are not designed or suitable for long-term home use or daily wearability. Other seizure alerting systems may operate by detecting motion of the body, usually the extremities. Such systems may generally operate on the assumption that while suffering a seizure, a person will move erratically and violently. For example, accelerometers may be used to detect violent extremity movements. However, depending upon the type of seizure, this assumption may or may not be true. Electrical signals sent from the brain during some seizures may be transmitted to many muscles simultaneously, which may result in muscles fighting each other and effectively canceling out violent movement. In other words, the muscles may work to make the person rigid rather than cause actual violent movement. Thus, some seizures may not be consistently detected with accelerometer-based detectors.

Ambulatory devices for diagnosis of seizures are generally not suited to grade seizures based on intensity, nor are they suited to differentiate seizure-related signals based on event type. Rather, different types of seizures and related events may often be grouped together. Accordingly, ambulatory devices for seizure detection may be ill-suited to customize responses for different types of detected seizure events. In addition, other ambulatory devices may not be ideally suited for cost-effective monitoring of some patients. For example, using current ambulatory devices, caregivers may misdiagnose some conditions, including, some that may benefit from condition-specific therapies. For example, some events, such as psychogenic or non-epileptic seizure events, may be grouped together with generalized tonic-clonic seizure events. Statistical analysis of event signals and pattern recognition methods may encourage effective diagnosis of some commonly misdiagnosed conditions. However, other ambulatory detection systems are generally not configured to provide organized statistical information to caregivers or process data for identification of specific activity patterns as may be used to medically or surgically manage a patient's care.

Accordingly, there is a need for epileptic seizure detection methods and apparatuses that can be used in non-institutional or institutional environments without many of the cumbersome electrodes to the head or extremities. There is further a need for detection methods that are suited to grade seizures by type and/or intensity, identify specific patterns of seizure or seizure-related activity, and customize alarms so as to provide robust and cost effective patient care. There is also a need for monitoring systems that organize medical data within databases to help medically and surgically manage patient care.

### SUMMARY

In some embodiments, a method or apparatus of monitoring a patient for seizure activity may include identifying peaks included among a collected EMG signal and calculating one or more peak property values for the identified peaks. The property values may be compared to qualification thresholds, the qualification thresholds including at least one qualification threshold associated with individual peaks and at least one aggregate qualification threshold associated with a group of peaks. Peaks meeting the qualification thresholds may be deemed to be qualified. And, once peaks are qualified, a level of qualified peak activity may be calculated and used in initiating a system response.

In some embodiments, a method or apparatus of monitoring a patient for seizure activity may include collecting an EMG signal and identifying peaks of elevated EMG signal. Peak data may be searched for one or more groups of peaks indicative of seizure activity by constructing various test groupings of peaks, calculating one or more property values for each of the test groupings, and comparing the property values to one or more threshold property values. Peaks may be qualified if they are part of at least one test grouping that meets at least one of the threshold property values, and from qualified peaks or bursts a level of activity may be determined and compared to a threshold level of activity for initiating a system response.

### BRIEF DESCRIPTION OF THE DRAWINGS

FIG. 1 illustrates embodiments of a method for qualifying peak data.

FIG. 2 illustrates model EMG data including a set of peaks and several groups constructible from the set of peaks.

FIG. 3 illustrates additional model EMG data.

FIG. 4 illustrates additional model EMG data.

FIG. 5 illustrates a bar distribution graph associated with model EMG data.

FIG. 6 illustrates embodiments of methods for combining groups of peaks.

FIG. 7 illustrates two groups of peaks in a model EMG data set.

FIG. 8 illustrates embodiments of a method for detecting or searching for seizure patterns in EMG data.

FIG. 9 illustrates embodiments of a seizure detection system.

FIG. 10 illustrates embodiments of a detection unit.

FIG. 11 illustrates embodiments of a base station.

### DETAILED DESCRIPTION

The apparatuses and methods described herein may be used to detect abnormal motor muscle activity, including, for example, seizure activity, and timely alert caregivers if activity is identified. The apparatuses may include sensors disposed on, near, or underneath the skin of a patient or attached to a patient's clothing and may be configured for measurement of muscle electrical activity using EMG. In some embodiments, apparatuses herein may include one or more processors suitable to receive an EMG signal and process the signal to detect seizure activity. Detection of seizures using EMG electrodes is further described in, for example, Applicant's U.S. Pat. Nos. 8,983,591 and 9,186,105. Applicant's U.S. patent application Ser. Nos. 14/920,665 and 14/816,924. Applicant's International Applications PCT/US14/61783 and PCT/US14/68246, and Applicant's U.S. Provisional Patent Application Nos. 61/875,429, 61/894,793, 61/910,827, 61/969,660, 61/979,225, 62/001,302, 62/032,147, 62/050,054, 62/096,331, and 62/149,434 the disclosures of each of which are herein fully incorporated by reference. Some of the methods disclosed in the aforementioned references describe detection of peaks of elevated EMG signal amplitude and qualification of peaks most likely to be related to seizure activity. Peak detection methods, including some which may be used in some of the embodiments, herein, are described, for example, in detail in Applicant's application Ser. No. 14/920,665.

In this disclosure, methods of detecting and qualifying peaks in a collected EMG signal, are also described. In addition, embodiments are described where groups of peaks may be constructed from among an initial group of peaks. Test groups of peaks may, for example, be compared against various qualification criteria or thresholds to facilitate qualification of peaks, in aggregate or combination, that may be related to seizure activity. Some of those embodiments may be particularly useful in detection or identification of peak combinations related to particular patterns of motor manifestations, including, for example, patterns associated with seizure activity that may otherwise fail to be identified in data that may be noisy or where data quality may be intermittent or sporadic. In addition to embodiments for real-time patient monitoring, embodiments herein may also be used to organize collected EMG data for use in databases of stored medical data.

In some embodiments, methods of monitoring a patient may include testing various groupings of peaks and searching for one or more groups that meet one or more qualification criteria. Qualification criteria may be associated with and tailored to identify particular patterns of seizure activity. In some embodiments where multiple groups of peaks may be constructed, groups may be combined in various ways to determine an appropriate response. For example, several groupings of peaks may be constructed each of which may, in some cases, independently meet a qualification criterion. An overall level of qualified peak activity may then be determined using procedures as further described herein. For example, in some embodiments, all peaks that are members of at least one group of peaks that meets qualification may be included in determining an overall level of qualified peak activity. An overall level of qualified peak activity may then be compared to one or more thresholds. Based on that comparison, an appropriate response may be initiated.

In some embodiments, methods of monitoring a patient may include testing multiple groups of peaks and searching for one or more groups that best fit one or more qualification criteria. For example, one or more groups may be selected which are characterized as having property values that meet qualification with greatest confidence and which may be most strongly correlated with seizure-related activity. In some embodiments, one or more property values for a group of peaks may be minimized or maximized in order to select or find peak groupings that may be related to seizure activity with high confidence or to find peak groupings most likely to be related to true physiological activity of a patient and not biased by inadvertent inclusion of peak data from sources of noise.

In some embodiments, qualification of peak data may be executed in steps or stages. For example, peaks may be initially qualified, peaks that fail initial qualification removed from further qualification, and remaining peaks organized in one or more groups. The groups may then be qualified based on comparison of group property values to aggregate property thresholds.

In some embodiments, properties and/or qualification threshold values may be selected for use in routines in order to provide or enhance selectivity for identification of particular patterns of seizure activity including, for example, clonic-phase seizure activity, psychogenic non-epileptic seizure activity, other activity patterns, and combinations thereof. Where reference is made to a routine that may be selective for identification of a specific activity, the routine may provide a positive response in a patient experiencing that activity, but the routine may provide a negative response in the absence of such activity, even if other types of seizure activity may be present. Therefore, execution of one or more selective routines may encourage identification of a particular type of seizure activity, such as one or more particular part or portion of a seizure. Routines herein that may be configured for detection of clonic-phase activity may be used to differentiate detected clonic-phase activity from other phases of seizure activity, including, for example, tonic-phase portions of seizure activity. Some of those routines may additionally be configured to differentiate detected clonic-phase activity from other activity commonly confused with the clonic phase of a generalized tonic-clonic seizure, including, for example, activity resulting from non-epileptic psychogenic seizure events.

At a high level, procedures for qualification of peaks may include comparison of various peak properties to one or more qualification thresholds. For example, if, for a peak, one or more peak property values meet one or more qualification thresholds, a qualification criterion may be deemed satisfied, and the peak may then be referred to as a qualified peak. Where a peak is qualified as meeting a criterion selective of clonic-phase seizure activity, the peak may also be referred to as a qualified-clonic-phase burst.

Some qualification procedures may operate on individual peaks. For example, certain properties of a peak such as its height, area, or duration width may be defined without including data from other peaks. Therefore, individual values for the property may be calculated for each peak in a group and compared to appropriate qualification thresholds. Properties of individual peaks may include, for example, peak height, peak area, signal-to-noise ratio (SNR) (e.g., a ratio of peak amplitude to estimates of uncertainty in peak amplitude as may be measured or estimated from background regions of signal), duration width, duration of intervening periods of lesser signal on either side of a trailing or leading edge of a peak, other properties of individual peaks, and combinations thereof.

In some embodiments, peaks may be compared against qualification thresholds selected from a group of qualification thresholds including a minimum duration width, maximum duration width, minimum signal-to-noise ratio (SNR), minimum duration of one or more intervening periods of lesser signal on either side of a peak, maximum duration of one or more intervening periods on either side of a peak, and/or combinations thereof. An intervening period may be defined by the duration length of a region of signal stability or lesser signal amplitude than elevated portions of a peak (e.g., low signal variability, RMS noise or signal magnitude) which may, for example, be marked by the distance between a peak edge and a nearby region of signal increase in magnitude or decrease in signal stability. In some embodiments, a signal-to-noise ratio for a peak may be calculated using amplitude data for the peak and an estimate or calculation of signal noise. Noise may, for example, be determined by calculating or estimating a level of variation or uncertainty in a baseline signal (e.g., uncertainty in measurement of signal amplitude, height, or area that may result from fluctuations in EMG data for a region not included in the peak) which may, for example, be determined from data collected on either side of a peak or from a separately measured portion of an EMG signal such as a portion collected when a patient is at rest. To calculate noise, for example, signal may be collected and signal variability may be directly measured. Alternatively, noise may, for example, be estimated from a signal magnitude and an estimate of variability expected from variations typical of a signal of that magnitude as predicted by one or more model functions, including for example, a normal distribution model function. In some embodiments, an estimate of variations or uncertainty in a baseline signal or noise may be selected or calculated during one or more system calibration routines.

In some embodiments, a peak may be qualified by meeting a threshold SNR, by meeting a minimum threshold for peak duration width of about 25 to about 75 milliseconds, and by meeting a maximum threshold for peak duration width of about 250 milliseconds to about 500 milliseconds activity. In some embodiments, a peak may be qualified based on the presence of an intervening sequence of lesser signal on either side of a peak of about 50 milliseconds to about 300 milliseconds. Other embodiments of peak qualification, including those that may operate on individual peaks, are further described in various others of Applicant's copending applications incorporated by reference herein.

Some properties of peak data may be calculated for more than one peak. And, in some embodiments herein, procedures for qualification of peaks may include comparison of a plurality of peaks to one or more qualification thresholds. That is, a plurality of peaks may be selected together as a group, an aggregate property value for the group of peaks determined, and the aggregate property value compared to one or more associated thresholds. If accurately characterized, aggregate properties of a plurality of peaks may be highly selective for seizure activity. However, many aggregate properties may be biased by the inadvertent inclusion of data from noise Sources and/or biased from inadvertent exclusion of desired data assoeiated with relevant physiological events. Many of the methods herein address this concern. For example, some embodiments herein may be used to find particular combinations of peaks indicative of seizure activity from among noisy data.

A qualification threshold value related to a property of a group of peaks may be referred to as an aggregate qualification threshold value. For example, included among aggregate qualification threshold values that may be used to qualify a plurality of peaks are minimum and for maximum rates of peak repetition, thresholds for variations in duration of times between peaks, other aggregate property thresholds, and combinations thereof. Aggregate qualification of groups of peaks is further described in Applicant's copending U.S. application Ser. No. 14/920,665 incorporated herein by reference.

In some embodiments, a group of peaks may be qualified against a threshold value for minimum repetition rate of peaks of about 1 peak per second and a threshold value for maximum repetition rate of peaks of about 7 peaks per second. In some embodiments, for example, if a greater number or lesser number of peaks than bounded by the above thresholds is present over an appropriate interval (e.g., an appropriate interval to scale a number of peaks as a peak rate), it may be deemed that the peaks may not meet qualification.

Included among various metrics for characterizing variation in duration of times between peaks is an average deviation percentage. However, other metrics for characterizing variability of peak timing such as standard deviation, average deviation or percentage deviation values may also be used. Any of the aforementioned metrics may be calculated and may be used as aggregate property values comparable to aggregate property threshold values as described herein. In some embodiments, a group of peaks may be qualified if an average deviation percentage value for time between peaks is greater than about 1% or about 5%. That is, an aggregate property threshold value of minimum average deviation percentage may, in some embodiments, be between about 1% to about 5%. In some embodiments, a group of peaks may be qualified if an average deviation percentage value for time between peaks is less than about 40% or about 50%. That is, an aggregate property threshold value of maximum average deviation percentage may, in some embodiments, be between about 40% to about 50%. Routines for determining variations in duration of times between peaks are further explained in greater detail in various others of Applicant's copending applications incorporated herein by reference.

In some embodiments, a procedure for peak qualification may include an initial qualification step based on one or more criteria as described above (e.g., criteria based on individual peaks), removal of peaks that fail the initial qualification, and another qualification step based on calculation of one or more aggregate property values for remaining peaks (e.g., all peaks that meet the initial qualification). For example, peaks may be identified, some peaks removed from overall qualification (e.g., peaks may be removed because the peaks, are too narrow or too wide), and then remaining peaks qualified if the remaining peak data as a whole meets one or more aggregate threshold criteria.

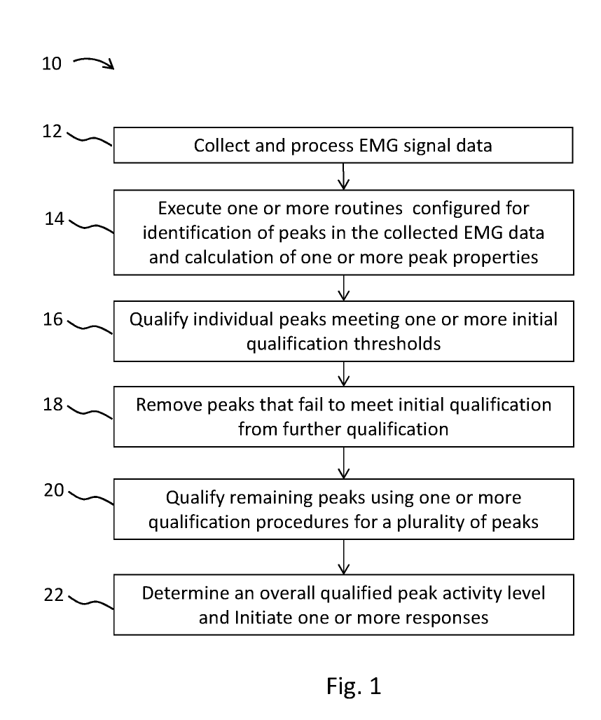

FIG. 1 illustrates embodiments of a method 10 for qualification of data including an initial qualification step based on one or more criteria of individual peaks and further qualification of data based on one or more criteria for a plurality of peaks in a group.

In step 12, EMG signal may be collected and processed. The signal may be processed using various techniques as may be used to improve detection of seizure activity and/or to condition the signal data for further analysis. For example, collected signal may be processed using one or more low-pass, high-pass, notch, or other filters to improve discrimination of seizure signals from other signals including sources of noise. In some embodiments, signal may be processed by removing high frequency signal components, such as by removing components above about 120 Hz or about 240 Hz. In some embodiments, one or more frequency hands may be isolated from a collected EMG Isolation of one or more frequency bands may include use of one or more filters and/or execution of one or more other procedures for isolation of signal data, including, for example, execution of a Fourier transform. In some embodiments, a plurality of frequency bands may be isolated from a collected EMG signal, and a T-squared statistical value may be calculated from the isolated signal data. In some embodiments, signal may be conditioned to prepare the data for processing using one or more peak detection programs, or signal may be conditioned for wavelet analysis. For example, signal data may be rectified, smoothed, and/or both.

In step 14, one or more routines may be executed that may be configured for identification of peaks in the collected EMG signal. Identification of peaks may, for example, include identifying portions of EMG data or portions of smoothed EMG data where curvature of the data changes (e.g., inflection or other critical points may be identified in the data). Trailing and/or leading edges of one or more peaks may be determined, which may be used to define temporal boundaries of peaks. Various peak characteristics or properties may then be determined. For example, one or more peak widths, peak height values, peak areas, or other peak properties may be calculated. Reference may be made to a peak amplitude, which as used herein may refer to either of a peak area or peak height unless where further specified. If peaks are successfully qualified, those properties may, for example, be used to determine a level of qualified peak activity.

In some embodiments, processing of EMG signal and/or peak detection (steps 12, 14) may include execution of smoothing techniques (e.g., moving average filter, Savitzky-Golay filter, Gaussian filter, Kaiser Window, various wavelet transforms, and the like), baseline correction processes (e.g., monotone minimum, linear, loss normalization, moving average of minima, and the like) and peak-finding criteria (SNR, detection/intensity threshold, slopes of peaks, local maximum, shape ratio, ridge lines, model based criterion, peak width, and the like) and may involve processing of rectified or unrectified data.

In some embodiments, signal may be processed and peaks detected (steps 12, 14) without loss or significant loss of temporal resolution of a collected signal. For example, data may be smoothed, integrated or both, but levels of processing may be appropriate to detect and qualify peaks as associated with clonic-phase activity. For example, protocols for smoothing and integration of data may be selected in order to maintain temporal resolution suitable for reliable detection of whether a peak meets selected duration width thresholds that are indicative of clonic-phase activity.

In step 16, peak data may be initially qualified against one or more qualification thresholds associated with individual peaks. As described above, various qualification thresholds may be used to qualify individual peaks. For example, in some embodiments, peaks may be qualified by meeting a threshold SNR and by meeting a minimum threshold for peak duration width of about 25 milliseconds to about 75 milliseconds and a maximum threshold for peak duration width of about 250 milliseconds to about 500 milliseconds.

In step 18, peaks that fail to meet initial qualification may be removed. Accordingly, step 18 may act as an initial screen to filter peaks from being included in other qualification steps. In step 20, remaining peaks that were not removed following step 18 may be qualified by calculating one or more aggregate properties of a plurality of peaks and comparison of property values to one or more aggregate property thresholds. For example, any of the above aggregate properties of peaks, including, for example, repetition rate or variability in times between peaks, may be used to qualify a plurality of peaks together as a qualified group.

Once peak data is qualified (e.g., qualified as including some number of qualified peaks), a level of qualified peak may then be determined. For example, following qualification of remaining peaks (as shown in the step 20), a level of activity may be determined and compared to an activity level threshold. As shown in step 22, a comparison of an activity level to a threshold activity level may be executed. Based on that comparison and/or other information, at least in some embodiments, a decision on whether to initiate one or more responses may be made. For example, a qualified peak count or rate may be determined and compared to a threshold qualified peak count or rate in order to determine if a response, including, for example, an alarm response, is warranted. In some embodiments, a response may include logging a positive detection of seizure activity, and if several consecutive or nearby positive detections are made, an alarm response may be initiated. In some embodiments, other responses or actions, in addition or as an alternative to an alarm response, may be initiated based, at least in part, on a level of qualified peak activity. For example, it a certain rate of qualified peak count is identified in a to part of a collected EMG signal, the part may be flagged or marked as a possible seizure event, the determined qualified peak rate may further be linked to that part of the collected signal data. The data and other associated qualified peak metrics may then be stored and included in a searchable database of medical data.

Steps in the method 10 may be executed within one or more time windows that may be of the same or different durations. For example, data may be collected and processed (step 12) over some number of suitable collection time windows as may be appropriate to maintain a desired temporal resolution for a monitoring system. Steps 14, 16, and 18 may generally operate as individual peaks are detected or at other suitable times so that those steps may be complete before remaining peaks are processed in step 20. Timing windows for group qualification (step 20) and for determining overall qualified peak activity levels and response initiation (step 22) may be adjusted for different routines. Generally, qualification time windows applied in step 20 and response windows applied in step 22 may be suitable in duration length in order to gather and analyze a desired amount of statistical data in order to reliably detect the presence of a seizure or seizure-related pattern. For example, in some embodiments, qualification time windows and response time windows may last for a duration of about 1 second to about 10 seconds or longer in some cases. For example, some patterns related to recovery from seizure activity or patterns indicative of non-epileptic psychogenic seizure events may be detected by examining signal collected over at least somewhat longer time scales than may be used for detection of initial seizure activity.

In some embodiments, all remaining peaks may be qualified together in a given window for qualification in step 20. For example, all remaining peaks within a qualification time window may meet or fail to meet an aggregate threshold value. Thus, all of those remaining peaks may be qualified and counted as qualified peaks or all of the remaining peaks may fail to meet the aggregate threshold value thereby failing overall qualification. Accordingly, those peaks may not be counted towards an overall level of qualified peak activity in step 22.

In some embodiments, aggregate threshold values may be selected to accommodate for some probability that one or more physiological events may be missed within a qualification time window. For example, aggregate qualification thresholds may be broad enough so that even if some number of physiological events (e.g., individual physiological events that tend to produce a seizure-related peak) fail to be detected, that other appropriately detected events do not fail aggregate qualification. For example, if, in a series of 8 adjacent physiological events associated with a burst of muscle activity in the clonic phase of a seizure, 7 peaks are detected, but a peak detection program fails to identify one event, the missing event may not skew the aggregate peak data so much that all peaks will fail aggregate qualification.

In some embodiments, a qualified peak activity level may be calculated in a response window that may extend over some number of adjacent or overlapping qualification time windows. For example, a response window may extend over a time frame that includes several qualification time windows. In some embodiments, successive or adjacent response time windows and/or qualification time windows may, for example, be overlapped to avoid or minimize latency between detection of seizure activity and initiation of a response as understood in the art. Response windows may also be tailored for a given pattern. For example, two or more routines may run simultaneously, the routines being configured or optimized for detection of different patterns and set to analyze data in response windows of different duration lengths.

Therefore, it should be understood that qualification time windows and response time windows may conveniently be of the same duration, but this need not be the case. For example, in some embodiments, a qualification time window may last about 2 seconds and adjacent qualification time windows may overlap for about 0.5 seconds. In each qualification time window, peaks may be qualified and some number of qualified peaks determined. A level of qualified peak activity may, for example, be determined at the completion of each qualification tune window and may, for example, include data from a preceding group of three qualification time windows (e.g., 5 seconds of data). If a calculated level of qualified peak activity exceeds a threshold, an appropriate response may be initiated. Because more than one qualification time window may be included in calculation of a qualified peak activity level, a risk that one or more spurious peaks may eliminate all peak data from aggregate qualification (step 20) may accordingly be reduced.

As described above, in some embodiments, qualified peak activity thresholds and/or other qualification thresholds may be broad enough so that even if some number of physiological events fail to be detected (or if a limited number of erroneous peaks are incorrectly counted), an alarm or other appropriate response may still be initiated. In some embodiments, an estimate of an actual number of peaks resulting from physiological manifestations of motor activity during a seizure may be made, and that number may be processed to have peaks that may be unrelated to seizure activity, such as noise spikes, removed. Because spurious events may be removed, thresholds for alarm initiation may, in some embodiments, be centered more narrowly around an expected range, including, for example, a range based closely on numbers of physiological events typically expected during a seizure. In some embodiments, thresholds for real-time detection of seizure events may be configured to improve sensitivity for seizures, but for other purposes, including, for example, meta data creation and/or linking of peak statistical information to collected data (e.g., for archiving in a searchable database), processing routines including different criteria may be applied. In some embodiments, routines for removing peaks expected to be spurious may be executed as part of archiving or retrieving data from a searchable database. Therefore, in some embodiments, some routines or combination of routines, including some that may have different threshold settings, may be executed as part of real-time seizure detection methods, data archiving methods as may be used, in creation of searchable databases, or both.

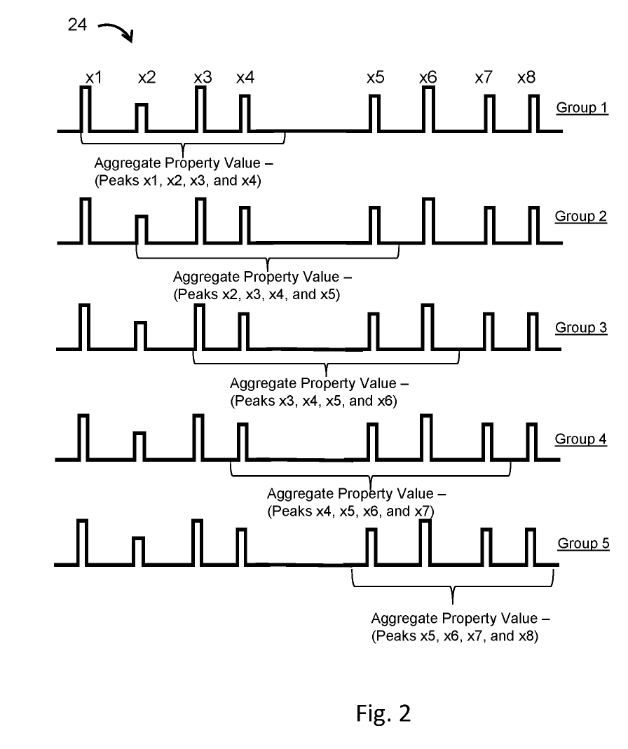

In some embodiments of methods for real-time seizure monitoring and/or methods of organization of data for a database, one or more test groups of peaks may be constructed, each of the one or more test groups of peaks being a subset group of an original group of peaks. As used herein, a subset of peaks or subset group of peaks is a portion of another set of peaks if every peak among the subset of peaks or subset group of peaks is a member of the other set. In some embodiments, one or more aggregate property values may be determined for each of multiple test groups that are a subset of an original set of peaks. Some of those embodiments may further determine if any of the subset test groups possess an aggregate property value that meets an aggregate property value threshold. For example, a test group of peaks constructible from an original set of peaks may include some number of consecutive peaks in the original peak set. For example, FIG. 2 illustrates various combinations or groupings of peaks that may be used as test groups of peaks and calculations that may be included as part of a qualification procedure.

In some embodiments, test group construction may be integrated together with the method 10. For example, as an alternative to embodiments of method 10 wherein a processor may only calculate one or more aggregate property values for all remaining peaks in step 20, one or more test groups of peaks may be constructed from a remaining group of peaks and 0110 or more aggregate properties for each of the one or more test groups may be compared against one or more aggregate property thresholds.

Referring again to FIG. 2, model data 24 is shown. The model data 24 may, for example, be produced in a scenario where initial qualification of peaks (as may, in some embodiments, be executed as described in step 16) and removal of peaks (as may, in some embodiments, be executed as described in step 18) provide 8 remaining peaks (x₁, x₂, . . . , x₈) within a time interval. Test groups may be created from those 8 remaining peaks. For example, peaks may be ordered based on a time stamp of when they were identified, and a first test group, including the first 4 consecutive peaks (x₁, x₂, x₃, x₄) in the time interval shown in FIG. 2, may be created. As part of qualification, it may be determined if one or more aggregate property values for that first test group meets one or more aggregate property threshold values. More generally, in some embodiments, some routines may determine if one or more aggregate property values for a set of test groups, each test group member of the set including some number of consecutive peaks (such as four of more) in the original set of peaks from which the test groups were created, meets one or more aggregate property threshold values. Referring back to the specific example for the model data 24, other test groups may also be created, and again aggregate property values may be determined and tested against thresholds. For example, a next group of 4 consecutive peaks (x₂, x₃, x₄, x₅) in the time interval may be constructed and used in a second aggregate property value calculation. In that next group, for example, peak x₅ was added and peak x₁ was removed. Further test groupings may be created and associated calculations determined as shown in FIG. 2. For example, for model data 24 a series of 5 consecutive groups may be created and aggregate property values determined and tested against an appropriate threshold for each created group.

In some embodiments, test groups may, for example, be created by incrementally selecting a next peak present in an interval and keeping the number of peaks in the test group the same. In some embodiments, aggregate property value calculations may be executed consecutively or in some other order so that all combinations of test groups in an interval that may include some number of consecutive peaks are considered. In each calculation, an aggregate property value for the constructed test group may, for example, be determined and compared to a threshold aggregate property value.

In some embodiments, test groups including other numbers of peaks (e.g., different than 4) may be constructed. Test group construction may include scanning a time stamped list of peaks in order of when they were detected and selecting some consecutive number of peaks to be included in a test group. The list may then be incremented and a next group among the list selected. For example, in FIG. 2, the groups 1-5 may be constructed by incrementing an ordered list in single units. In some embodiments, a list of peaks may be incremented in other desired units. Once a set of test groups is constructed, property values for members among the set of test groups may be calculated, such as in a convenient order, and peaks may be identified that meet qualification for at least one of the test groups. For example, in some embodiments, a peak may be deemed qualified if it is part of at least one test group that meets qualification. For example, peaks in a window may be treated as described above in groups of 4 or some other suitable number such as about 4 to about 8 peaks. Such procedures may, for example, be used to limit a number of calculations executed in processing data. In addition, it may be more convenient to write protocols executed by a computer processor to construct test groups including some pre-selected number.

In some embodiments, in addition to use of a certain pre-selected number of peaks for creation of test groups, other test groups may be created including test groups including other numbers of peaks or including test groups that include a range of peak numbers. Accordingly, another set of calculations of aggregate property values may be executed. For example, one aggregate property value may be calculated for each group. More than one aggregate property value may also be calculated for each group.

To identify particular patterns, different combinations of aggregate properties and aggregate property thresholds may be applied. Continuing with the example herein where groupings of 4 consecutive peaks are considered, aggregate property values may next be determined for groups of 5 consecutive peaks. In some embodiments, groups of peaks may be limited to those that may fit within a preselected time window. In other embodiments, one or more test group of peaks may wrap or extend into an adjacent window. Appropriate rules may be established for specific routines to deal with test groups that may extend into an adjacent window. Referring to the example in FIG. 2, and where only combinations of consecutive peaks that fit in the time window are considered, such as for ease in explanation, to consider all combinations of test groups that include 5 consecutive peaks, 4 test groups may be created. That is, a first group of 5 consecutive peaks in this example may include the peaks (x₁, x₂, x₃, x₄, x₅). Other groups, including a last group in the set (when iterating forward in the time stamped data in single peak increments) including the peaks (x₄, x₅, x₆, x₇, x₈), may be created.

In some embodiments, a group of peaks ranging from some minimum number of peaks (e.g., a chosen minimum number to qualify as a group with a suitable number of peaks to perform desired calculations—such as a number to reliably calculate a standard deviation for times between peaks) to the total number of peaks identified in a time window may be examined. For example, selecting 4 peaks as a minimum number of peaks that may be suitable to accurately qualify peaks using a certain aggregate property, if over a 2 second period of time 8 peaks were identified, various groupings including a number of 4, 5, 6, 7, and 8 peaks per group may be constructed and included in qualification calculations. In that case, by way of example only, a total of 15 groups may be constructed.

In some embodiments, test groups may consist of peak groups including only consecutive peaks in a group of peaks from which test groups may be constructed. In other embodiments, test groups may include consecutive peaks, non-consecutive peaks (e.g., peak combinations where one or in some cases, more than one intervening peak that is excluded from the test group), or both.

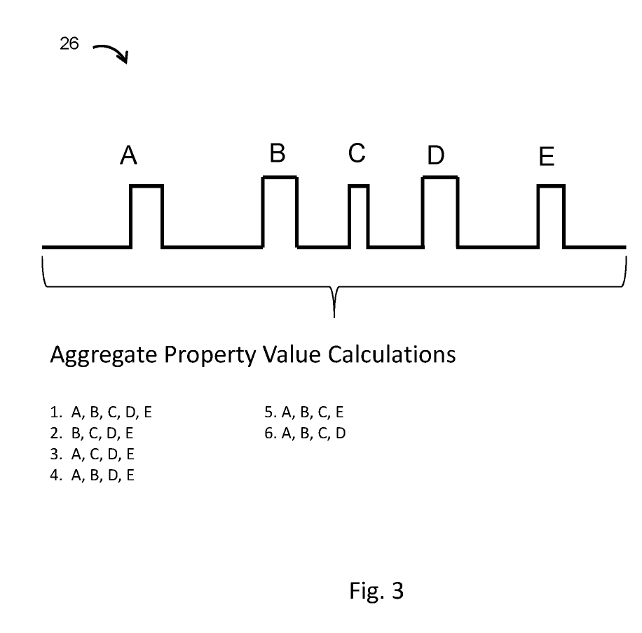

For example, FIG. 3 shows model data 26 and various groups of peaks that may be constructed. Model data 26 may, for example, be produced in a scenario where initial qualification of peaks (as may, in some embodiments, be executed as described in step 16) and removal of peaks (as may, in some embodiments, be executed as described in step 18) provide 5 remaining peaks A, B, C, D, and F) within a time interval. Groups may be constructed from that group of remaining peaks. In the model data 26, a group of peaks (A, B, C, D, and E) is shown. The peak C may be a peak that is an artifact of noise and unrelated to the desired physiological signal intended for measurement in EMG, but may have failed to be removed by other processing methods. Groupings including non-consecutive peaks may work under an assumption that a spurious peak failed to be removed in initial qualification or in other protocols, and a procedure may test whether excluding a peak from a group of peaks results in identification of a group of peaks that may be qualified to be indicative of seizure or seizure-related activity. For example, when removing peak C, an expected pattern or group of peaks may result. For example, such a pattern may be characterized by meeting both of an aggregate threshold for repetition rate and variability of times between peaks. And, the peak combination (A, B, D, E) may be deemed to meet a criterion for a seizure pattern the seizure pattern, the seizure pattern being identified by removing one spurious peak from an initial group of peaks.

or some patients, particularly, if, for example, contact between the skin and electrodes has become poor, a condition that may, at least in some cases, be the result of excessive sweating and/or other conditions that may increase in likelihood as a patient transitions towards a seizure or during seizure recovery, one or more peaks not directly related to clonic-phase activity may fail to be removed by initial qualification or by other screening means. In that event, improved protocols for examining whether spurious peaks are present in a given data set may be advantageous for some patients and/or in some situations. Construction of test groups of non-consecutive peaks may meet the aforementioned need for improved protocols.

In some embodiments, if a number of peaks is removed from data as part of peak qualification, other requirements for Mann initiation or emergency alarm initiation may be used to minimize false positive detections. Or, a response may be different if peaks have been removed in order to meet an activity level threshold. For example, a detection protocol may identify a seizure related pattern by removing some number of peaks from a particularly noisy data set. The system may then initiate one or more warning protocols, including those that may or may not contact a caregiver or patient (e.g., the one or more protocols may terminate passively), and then examine data for one or more corroborating events, detection of which may improve confidence in seizure detection. Some procedures that remove peak data may also be executed as part of data archiving. For example, one set of routines may be used to initiate one or more alarm protocols. Another set of routines, including routines configured to remove suspect or erroneous noise peaks and look for seizure patterns, may be configured to isolate or find peak patterns indicative of true physiological activity and not biased by inadvertent inclusion of peak data from sources of noise.

In some embodiments, protocols for removing spurious peaks may work by removing one or more peaks without specific consideration for how likely it may be that the peak is erroneous or without specific consideration for whether a removed peak is more or less likely to be spurious than other peaks in a peak set. For example, in some embodiments, peaks may be removed from a remaining group of peaks serially or in some convenient order to test different combinations, but the carder in which peaks are removed may not be related to any specific property of a given remaining peak. However, in some embodiments, decisions or algorithms for removing peaks may be based on other factors. For example, qualification of peaks by determining an aggregate property for a plurality of peaks (e.g., as described in step 20) may include statistical processing to determine whether any peaks among the plurality should be removed because one or more peak properties indicates that the peak may be an outlier. For example, it may be found that including a given peak in a group inordinately biases a property value (or aggregate property value) such as variability for times between peaks. For example, a spurious peak may produce one or more data points in a property value calculation that bias group data in a manner greater than predicted for a normally or otherwise distributed data population. And, by removing that given peak from a group of peaks data that may otherwise fail qualification, a subset peak set may then be well qualified. Any of various mathematical techniques for processing data to determine the presence of outliers may, in some embodiments, be executed.

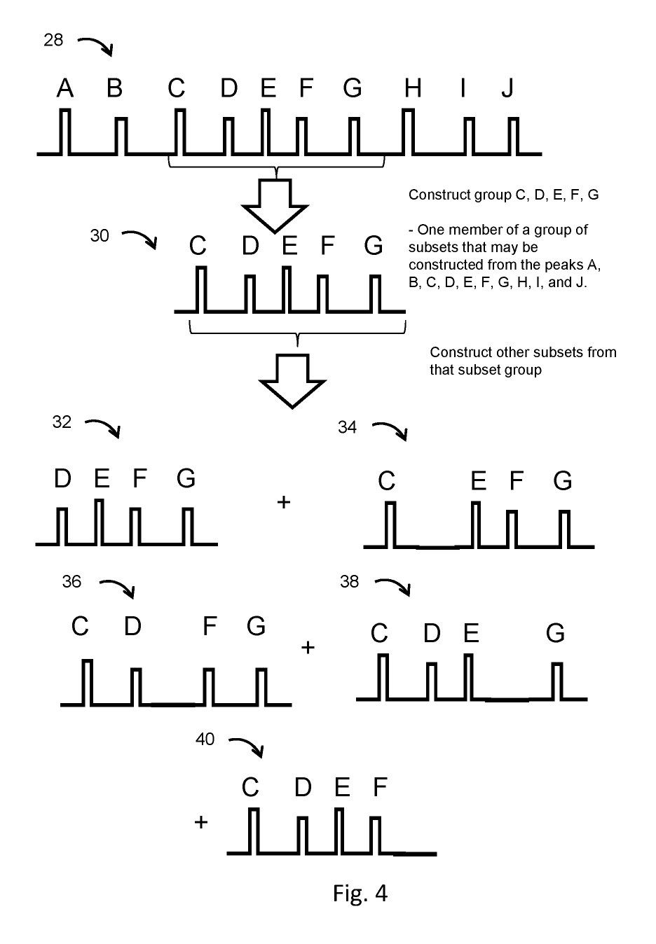

In some embodiments, test groupings of peaks may be made from a group of peaks that is itself a subset of another group. Embodiments where test groups may be constructed from other groups that are themselves subsets of another group of peaks may be understood in reference to FIG. 4. FIG. 4 shows a model set of peaks 28. Model set of peaks 28 may, for example, be produced in a scenario where initial qualification of peaks (as may, in some embodiments, be executed as described in step 16) and removal of peaks (as may, in some embodiments, be executed as described in step 18) provide 10 remaining peaks (A, B, . . . , J) within a time interval. A group of peaks 30, including the peaks (C, D, E, F and G), which is a subset of the remaining peaks (A, B, . . . , J) is shown. The group of peaks 30 may be constructed from the model set of peaks 28 as described, for example, in reference to FIG. 2. For example, the group of peaks 30 may be constructed by scanning a time stamped list of a model set of peaks 28 in order of when they were detected and selecting some consecutive number of peaks to be included in a subset group. The list may then be incremented to identify a plurality of groups, including the subset group 30 shown in FIG. 4. Other groups of peaks (e.g., groups 32, 34, 36, 38, and 40) may then be constructed from the subset group 30. For example, the aforementioned groups (32, 34, 36, 38, and 40) may be constructed by removing serially one peak from the group 30.

While serial removal of peaks from the subset group 30 may produce a limited number of test groups, other subset groups may be produced from model set of peaks 28. Those other subset groups may each serve as a basis for creation of test groups. Generally, based on constructing a set of subset groups, and then executing serial removal of one or more peaks from members of the set of subset groups, a significant number of test groupings may be constructed. Accordingly, the methods herein may be effective at searching a significant number of peak combinations and finding patterns of activity from noisy data. In some embodiments, only a suitable number of peaks, such as one peak, may be removed from a first subset group. In some cases, a maximum ratio of peaks that may be removed during construction of test groups may be defined in an algorithm. For example, in some embodiments, a maximum allowed ratio of removed peaks to peaks in a group remaining after peak removal may be about 1:5 to about 1:10. By selecting an appropriate number of peaks in a constructed subset group (e.g., 5 peaks as shown in FIG. 4) and/or a suitable number of peaks excluded or removed from those subset groups to generate test groups, multiple test groups may be constructed thereby increasing the chance that a particular pattern of peaks is constructed that will meet qualification. However, the number of possible peaks excluded from test groupings may be controlled. Accordingly, risk of false detection of a pattern may also be limited. And, therefore, in some embodiments, procedures may be made that are suitable for searching noisy data, but still minimizing risk of false positive detection, a problem that may otherwise be present if test groups are made randomly and/or using other protocols that may allow indiscriminate peak exclusion. Adverse consequences of false positive detection may be more problematic in methods associated with real-time detection of abnormal muscle activity. However, those concerns are not the same for a caregiver reviewing or searching data in a database. Accordingly, in some embodiments, it may be advantageous to execute different procedures for construction of test groups as may be used in organizing data for inclusion in a searchable database than used in real-time monitoring. For example, more aggressive algorithms for peak removal may be applied when including data in a searchable database.

In some embodiments, procedures for generating test groupings may include removal of peaks from an initial group of peaks wherein the removal of peaks is executed in a logical order or pattern. For example, a method of monitoring a patient may include construction of test groups wherein test groups most likely to be associated with a physiological pattern are tested first or ranked with higher confidence. For example, a procedure may order peaks based on one or more peak characteristics. Based on that ordering, a list of candidate peaks most likely to be spurious (e.g., not related to physiological activity) may be generated. For example, a group of peaks may be ordered based on duration widths and that ordering may be used to identify that the duration widths of a majority of peaks are clustered around a central range, but one or more peaks may be characterized as having a duration width outside of that central range. Peaks outside of the most common range may be removed, first removed in generation of test groupings, or test groups created from such removal may be ranked at higher confidence than other test groups. In some embodiments, test groupings may be constructed by removing one or more peaks at either end of a peak characteristic ordering. For example, the highest or lowest value in an ordering of one or more peak characteristics may be selected for removal.

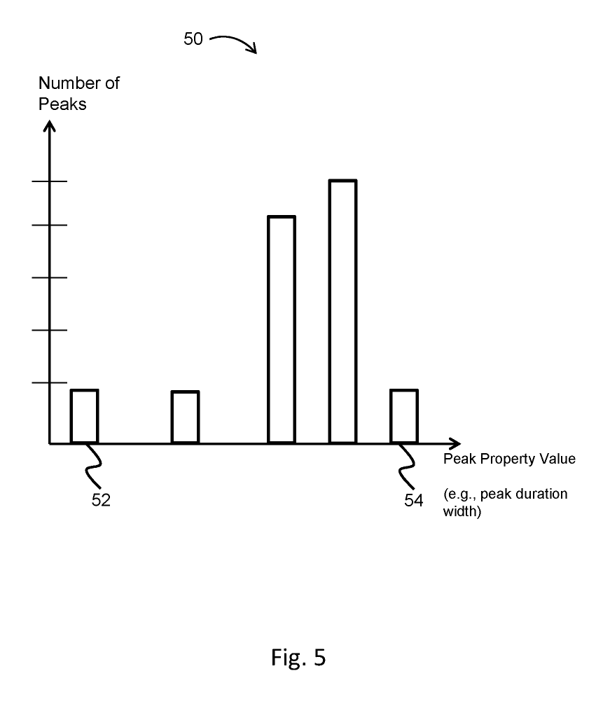

For example, FIG. 5 shows a model bar graph characterizing the distribution of peaks identified in a collection time period and shows bars 52, 54. Peaks associated with bars 52 and 54 are characterized as having a minimum property value 52 and a maximum property value 54 among a group of identified peaks. Peaks associated with those bars 52, 54 may be indicative of peaks with property values at the edge of initial qualification thresholds. As shown in FIG. 5, most of the peaks tend to be clustered towards greater values of peak duration. And, the peak associated with bar 52 shows the largest deviation from other peaks in the group. Therefore, that peak may be removed from the set of peaks or removed first in selection of a test group. Test groups produced by removing the peak associated with bar 52 may be viewed with higher confidence than other test groups associated with removal of other peaks. An aggregate property value for the subset of remaining peaks may then, for example, be determined. In some embodiments, a mean value for the initial group of peaks may be determined and a deviation value from the mean determined for each individual member among the initial group of peaks. Test groups may be made by removing peaks in order of those peaks identified as having the highest deviation from that mean. For example, as shown in FIG. 5, the peak associated with bar 52 may have the highest deviation from a mean value and may be most likely to be erroneous or related to noise in the collection window. Therefore, that peak may be removed or removed first in a protocol for generating test groups of peaks.

In some embodiments, a routine may remove peaks from another group of peaks based on how well peaks in the group meet an initial qualification condition. For example, a routine may assign an initial certainty value to peaks and test whether removing one or more peaks facilitates qualification and the identification of a seizure-related pattern. To minimize calculation resources or to provide other metrics of overall certainty, a routine may generally test or first, test if removal of peaks with the lowest certainty value results in identification of an identifiable seizure pattern (e.g., a group of peaks that meet an aggregate property value threshold). In some embodiments, once a seizure related pattern is identified, computing or signal transmission resources may be allocated to most efficiently execute one or more system operations. For example, warning or emergency messages may be issued as part of alarm initiation protocol, and further construction of test groups or transmission of data associated with test groups (e.g., as may be sent from a remote device to a managing device or base station) may cease or only be executed after completion of other operations, such as those most critical to make sure emergency care is provided to a patient. Certainty values of peaks may, in some embodiments, be based on patient or patient demographic values or based on agreement with other peaks collected during a given time window, in some embodiments, certainty values for peaks may be based on a combination of metrics including, for example, SNR, width and amplitude as described in more detail in Applicant's U.S. Pat. No. 8,983,591 incorporated herein by reference.

For any given time interval during a monitoring session, a number of detected peaks or initially qualified peaks may be determined, and for a majority of times within the monitoring session those numbers may generally be low. For example, even if a random noise event is erroneously identified as a peak, most time windows may only include a limited number of such events. Furthermore, normal movements unrelated to a seizure may generally not produce peaks or initially qualified peaks at a high rate. That is, using procedures herein for initial qualification of peaks, peak data from most motor manifestations associated with non-seizure movements may be removed.

In some embodiments, if a certain number of peaks or initially qualified peaks is identified, an algorithm may attempt to fit remaining peak data to a seizure pattern. For example, in some embodiments, only if a suitable number of peaks to generate test groupings is identified may test groups of peaks be constructed. For example, remaining peaks following step 18 in method 10 may, for a majority of intervals in a monitoring session, be low and accordingly a method may not need to execute further calculations associated with construction of test groups or execute multiple calculations of aggregate property values. Accordingly, in some embodiments, operations associated with test group generation and property value calculations may only be executed when necessary, and computational resources dedicated to those calculations and remote transmission of that data may further be limited. In some embodiments, including embodiments where operations associated with test group construction may be executed on one processor and then transmitted to another processor for execution of peak construction and/or comparison to multiple patterns, protocols for selective execution of test group construction may be applied.

In some embodiments, certain computations described herein, such as test group construction, may be automated, executed with any suitable processor among the various components described herein (e.g., detection units and/or base stations), and may only be executed if a threshold number of peaks or initially qualified peaks are found within a given time window, a condition that may generally be limited particularly if time windows, initial qualification protocols, and requisite remaining peak numbers (needed to invoke further calculations) are suitably chosen such as described in various embodiments herein.

In some of the embodiments herein, in a certain time period such as a response window, more than one group of peaks may be qualified. For example, such a scenario may arise when multiple peaks are identified from EMG data collected over a response window, multiple test groups are constructed over that time period, and different test groups are found to meet qualification. In some embodiments, peaks among different test groups may be distinct. In other embodiments, test groups may sometimes include peaks that are members of more than one group. And, once qualified groups of peaks are identified, those groups may be combined and used to calculate an overall level of qualified peak activity. Various methods of combining groups of peaks, determining levels of qualified peak activity, and initiating one or more system responses based on levels of qualified peak activity may be executed in the embodiments described herein. For example, techniques are described herein for calculating overall levels of peak activity where groups include only distinct peaks. Other techniques may be applied where individual peaks may be separately qualified in more than one group.

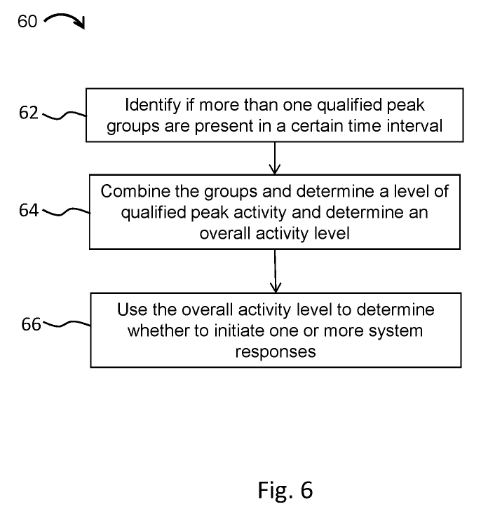

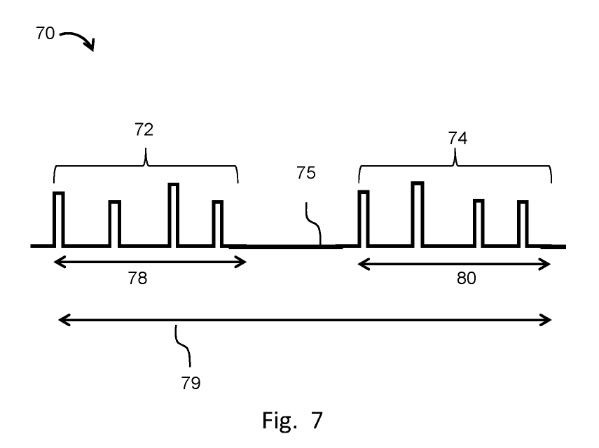

FIG. 6 illustrates embodiments of a method 60 for combining groups of peaks. For example, as may be understood in reference to the model set of peaks 70 (shown in FIG. 7), it may be found that when including the first 4 peaks therein in a first test group 72 that the first test group 72 may pass qualification. Thus, the first test group 72 may also be referred to as a first group of qualified peaks. A second test group of peaks 74 may likewise pass qualification and may be identified as a second group of qualified peaks. Accordingly, the model set of peaks 70 may be found to include 8 total qualified peaks present as two distinct groups 72 and 74. In this scenario, the two groups include distinct peaks. That is, no peak is a member of both groups. It should also be understood that the groups 72, 74 may be qualified even if including all 8 peaks may result in qualification failure. For example, if all peaks in the model set of peaks 70 were qualified by calculating a repetition rate for the peaks, the data set may be biased by the relatively long gap 75, which may be present if, for example, a peak finding routine failed to detect one or more abnormal motor manifestations during a seizure.

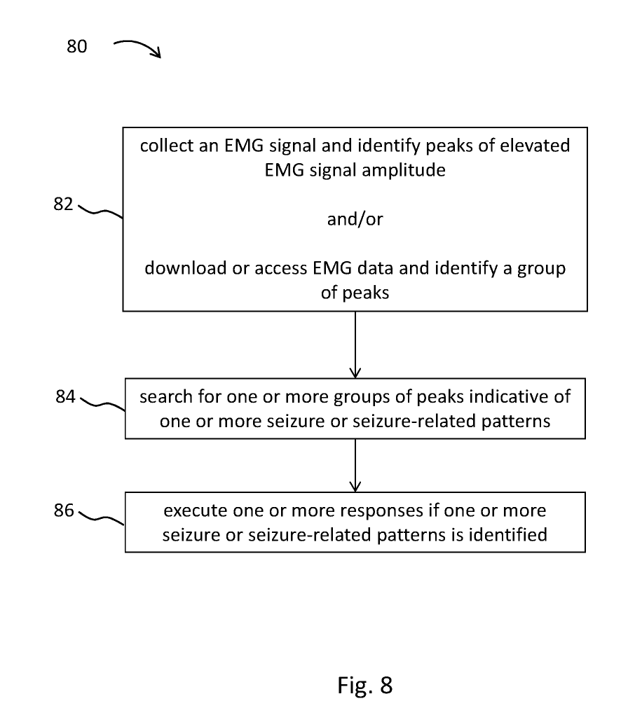

Referring again to FIG. 6 and FIG. 8, in step 62, it may be identified that the two groups 72, 74 meet qualification, and one or more techniques may then be used to combine the two groups. For example, in step 64, distinct groups of qualified peaks, such as groups 72, 74, may be combined by summing each of the peaks in the two distinct groups. An overall level of qualified peak activity may then be calculated. For example, for the set of peaks 70, where the groups 72 and 74 are separately qualified, 8 qualified peaks may be combined together and used to calculate an overall level of qualified peak activity. In step 66, a decision may be made if the overall activity level is above a threshold. For example, if a threshold activity level is exceeded, it may be determined that an alarm should be initiated.

In some embodiments, combining of more than one group of qualified peaks (step 64) may include comparing one or more property values for different qualified groups. For example, peak statistics may be calculated for each group of peaks, and a degree of similarity or difference for various peak statistics between the groups may be determined. Notably, for some patterns a degree of similarity between groups of peaks may be expected. For example, if the groups of peaks are part of an intermediate portion of the clonic phase of a generalized tonic-clonic seizure, the duration of periods adjacent an elevated peak portion may generally be of similar duration. Accordingly, if two or more groups of peaks show similarity in this statistical metric, it may be deemed that the groups in combination match an expected pattern for intermediate, portions of the clonic phase. For other patterns, a degree of difference in the aforementioned metric may be expected between groups of peaks collected over time. For examples normal progression out of the clonic phase of a seizure may be recognized if peaks across two or more groups in a response window are characterized by an increase in the duration of periods adjacent an elevated peak portion.

In some embodiments, combining of more than one group of qualified peaks (step 64) may include determination of one or more pooled property values based on peak data from among the various groups. For example, two or more groups of peaks may be separately qualified, and a pooled property value may then be calculated to determine a relationship, such as a degree of similarity, between the two or more groups. In some embodiments, if a pooled property value for a plurality of groups meets a threshold value, e.g. if the pooled property value is within a specified minimum and/or maximum threshold value, separate qualified peak groups may be combined, and an overall level of qualified peak activity may then be determined. Therefore, in some embodiments, independently qualified peak groups may be subject to a further step, of qualification before determining an overall peak activity value.

For example, two peak groups separated in time may be independently qualified, and by pooling property values together it may be estimated whether the two groups may reliably be treated as part of the same portion of a seizure. For example, in two separate duration intervals of an intermediate portion of the clonic phase of a seizure, two peak groups may be measured, but in an intervening section of data collected at times between the two intervals, data may be noisy, and that section of data may not produce a useful peak group. However, those two peak groups may each be separately qualified, and if pooling the data together indicates that the trains are each part of an intermediate part of the clonic phase, those two separate groups may be combined to determine an overall qualified peak activity level. Accordingly, that overall qualified peak activity level may be used in determining an appropriate response.

A pooled property value may refer to a property value calculated by including data from two or more peak groups weighted according to peak numbers and degrees of freedom. For example, in some embodiments, a property value for a group of peaks may be a variability of times between peaks as may be characterized as a standard deviation. To calculate a pooled property value for two groups, a first standard deviation values may be calculated for one group of peaks where n₁ is the sample size of the group. A second standard deviation value s₂ for the second group of peaks may also be calculated where n₂ is the sample size of that second group. A pooled standard deviation value including data from the two groups of peaks may then be calculated as follows:

S_{(pooled)}=Sqrt[[(n₁−1)S₁²+(n₂−1)S₂²]/[(n₁−1)+(n₂−1)−1]] Equation 1

More generally, for any number of groups of peaks n₁, n₂ . . . nₖ with standard deviation value s₁, s₂, . . . sₖ a pooled value may be calculated as follows:

S_{(pooled)}=Sqrt[[(n₁−1)S₁²+(n₂−1)S₂²+ . . . +(nₖ−1)Sₖ²]/[(n₁−1)+(n₂−1)+ . . . (nₖ−1)]] Equation 2

In some embodiments, a pooled value may serve as a check to verify that groups of peaks may logically be considered part of the same portion of a seizure. For example, generally in one part of a clonic phase, peak rates may be maintained within certain bounds even if there is a general lowering of peak rate during later stages of the clonic phase. And, by pooling data from multiple groups of separately detected and qualified peak groups, sporadically detected peak data in a noisy signal may still be combined with confidence.

FIG. 8 illustrates embodiments of a method 80 for collecting EMG signal, identifying peak data in the EMG signal, and evaluating whether one or more seizure or seizure-related motor manifestation patterns may be present. In some embodiments, method 80 may be executed in real-time as a patient monitoring strategy and EMG signal may be directly collected. In other embodiments, method 80 may be executed following signal collection, including, for example, when adding data to or searching data in a medical database. The various classes of embodiments are described, in the alternative, in step 82. For example, in step 82, EMG signal may be collected and peaks may be identified in the collected EMG signal. Alternatively, previously collected signal data may be accessed such as by downloading a portion of signal data.

In some embodiments, EMG signal may be collected, peaks detected, and an initial filter or screening of those peaks may be made before searching the data for the presence of one or more patterns. For example, individual peaks may be screened against one or more properties of the individual peaks prior to executing other operations. For example, in some embodiments, peak screening operations, such as step 16 and step 18 of method 10, may be executed as part of step 82. In some embodiments, where method 80 is executed from existing EMG data, an operator of a database may, for example, choose one or more patients and/or one or more time ranges of collected data. A program may subject the data to peak detection, or peaks already identified may be downloaded or accessed for further processing.

In step 84, method 80 may include searching identified peaks for one or more groups of peaks indicative of one or more seizure or seizure-related activity patterns. In some embodiments, step 84 may include two parts. For example, in a first part, an initial group of identified peaks may be subjected to test group construction. That is, multiple subset test groups may be made from the initial group of identified peaks. Second, property values of test groups may be compared to property value thresholds suitable to qualify a test group as indicative of a given pattern.

In some embodiments herein, various motor manifestation patterns may be detected, including, for example, patterns associated with epileptic seizure activity, progression throughout a generalized-tonic-clonic seizure, non-epileptic psychogenic seizure activity, and combinations thereof. In some embodiments, patterns of activity associated with post-ictal motor movements may also be identified. To detect or search for a pattern, peak data, including, for example, different groups of peaks constructed from an original group, may be compared to one or more qualification thresholds suitable to define the pattern. Patterns may also be defined by protocols used for test group construction. For example, some patterns may be identified using different algorithms for test group construction. For example, algorithms may be defined with rules that allow or do not allow for application of test groups with excluded peaks or that define a maximum number or ratio of excluded peaks.

In some embodiments, more than one pattern may be searched or identified in a collected EMG signal. For example, in some embodiments a first seizure pattern may be characterized by detection of a threshold qualified peak number of at least 8 qualified peaks included among no more than one test group. In another example, a seizure pattern may be characterized by detection of a threshold qualified peak number of at least 12 qualified peaks included among no more than two test groups. In another example, a seizure pattern may be characterized by detection of a threshold qualified peak number of at least 15 qualified peaks included among no more than three test groups. The aforementioned patterns may also be defined by one or more qualification thresholds or pooled property thresholds. For example, peaks in the aforementioned patterns may be qualified if groups of peaks exhibit a peak rate of greater than a minimum peak rate of about 2 peaks per second and less than a maximum peak rate of about 7 peaks per second. In some embodiments, the peaks may be defined by alternate or additional aggregate qualification car other qualification thresholds.

For example, peaks in the aforementioned patterns may be qualified against a minimum threshold peak duration, width of about 100 milliseconds and a maximum threshold peak duration width of about 300 milliseconds, an aggregate average deviation percentage value for duration of times between peaks of between about 3% and about 40%, or both.

In some embodiments, a seizure pattern may be characterized by detection of a threshold qualified peak number of at least 8 qualified peaks to at least 12 qualified peaks. The pattern may further be characterized by qualification thresholds or pooled property thresholds of a peak rate of greater than a minimum rate of about 5 peaks per second and less than a maximum rate of about 7 peaks per second. The aforementioned pattern may sometimes be part of a group of patterns. For example, another pattern may identify peaks in a range of between about 4 to about 6 peaks per second. Still another pattern may identify peaks in a range of between about 2 to about 5 peaks per second. Another pattern may identify peaks in a range of between about 1 to about 3 peaks per second. Still another pattern may identify peaks in a range of between about 0.2 to about 1 peak per second. Other qualification thresholds, including, for example, minimum or maximum, values for a required duration width of elevated portions of a peak, a required duration of one or more intervening periods of lesser signal on either side of elevated portions of a peak, a required average deviation percentage value for duration of times between peaks, and/or combinations thereof may also be included.

A particularly useful pattern may be defined where peaks are presented at a relatively low rate, such as less than about 3 peaks per second, and where thresholds of duration width of elevated portions of a peak and threshold duration width of periods of lesser signal on either side of a peak are used in peak qualification. Generally, such low repetition rates may be associated with times where a patient is recovering, either normally or abnormally, from the clonic phase of a seizure. For example, particular patterns, or combinations of patterns, may be defined that organize data so that it may be identified whether a patient generally or always transition out of a seizure with a sudden or gradual change in repetition rate of peaks. Patterns may further identify whether a change in repetition rate of peaks is linked with a certain change in the duration width of elevated portions of peaks, of width of lesser portions of signals between elevated portions of peaks, or ratio between elevated portions of peaks to lesser portions of signals between elevated portions of peaks. For example, if a patient tends to transition out of a seizure without experiencing an expected rate of increase in a duration width of lesser portions of signals between elevated portions of peaks that pattern may be flagged as possibly indicative of psychogenic non-epileptic seizure activity. In addition, qualification of groups based on a distribution of times between peak patterns may be created that show either of a gradual progression out of the seizure or a more sporadic pattern. Changes in amplitude of peaks may Wither be considered.

In some embodiments, more than one pattern indicative of seizure or seizure-related activity may be detected during a monitoring session for a patient. For example, in step 84, it may be determined that one seizure pattern is identified and later another pattern may be identified. Combinations of seizure patterns may be used to gain information that may be difficult to gather from other patterns or simpler patterns. In a related manner, in some embodiments, some simpler patterns herein may act as building blocks for more complicated patterns. For example, using the aforementioned patterns a more complicated pattern may be defined. Some of those patterns may last for significant durations extending throughout more than one part of the clonic phase of a seizure, including transition out of the clonic phase.

In some embodiments, different patterns that may be associated with a common clinical diagnosis may be grouped together. Accordingly, where one or more of those patterns is identified, the significance of that pattern may be flagged and presented to a caregiver. For example, generally, where transition out of a seizure is sudden and not accompanied by a change in duration of lesser periods on either side of peaks, such a pattern may be flagged as possibly related to an occurrence of psychogenic non-epileptic seizure activity. Patterns indicative of this behavior may be flagged even if they differ in other characteristics, such as average repetition rate or average duration of lesser periods on either side of peaks.

It should be understood that some patterns may include property value thresholds associated with individual peaks in addition to aggregate property thresholds. For example, it may be useful to compare peak data to individual property value thresholds as part of an initial screening used for generation of an initial peak set that may then be subjected to test group construction. It may also be useful to compare peaks during or after this initial screen to one or more individual property values. For example, during different parts of a seizure, some properties of individual peaks may be expected to change. Accordingly, the best screen for generating test groups suitable for use in examining one part of a seizure may not be the same as for generating test groups suitable for examining other parts of a seizure. Accordingly, in some embodiments, an initial broad screen of individual peak properties may be applied in order to define an initial group of peaks for test group construction. Further processing may include determining whether peaks in a test group possess individual property values that may be more stringent than applied in the initial screening of peaks.

Other patterns (e.g., in addition to the particular embodiments above) may also be stored in a database and may also be compared to collected data. Patterns may be defined to look at activity over a time window. For example, one pattern may look for a group of peaks that exhibit property values typically found near the start of the clonic phase of a seizure, and another pattern may look for a group of peaks exhibiting property values typically found near the end of a clonic-phase portion of a seizure. Windows suitable to look at those different parts of a seizure may be different. For example, windows may be made appropriate so that the peak data is limited to relatively brief time windows (e.g., from about 2 to about 10 seconds). Other patterns may consider data over longer durations and look for activity where peak activity changes over time as may, for example, be expected during seizure progression. Patterns that consider data over greater periods of time may look for the presence of independently qualified groups and look for trends in statistics of successive or nearby groups.

For example, a pattern may look for a first pattern set with a threshold typically achieved by activity at the start of a clonic-phase portion of a seizure and also look to see if that pattern repeats or if a second pattern associated with later portions of the clonic phase may also be present. The first and second patterns may, for example, have different thresholds for peak width, peak repetition, peak amplitude or combinations thereof, and in some embodiments, a pattern may be selected to see if those characteristics change or if, for example, activity terminates without a characteristic change in peak width, repetition and/or amplitude. And, in some embodiments, a caregiver may search data for patterns where qualified peaks change over time in order to assist the caregiver in identifying if the patient may be prone to non-epileptic psychogenic events.

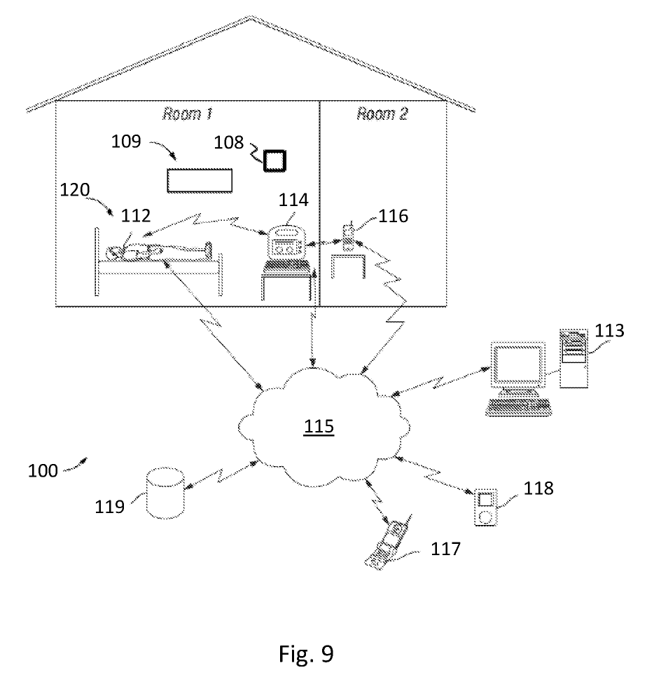

A variety of systems may be suitable for collecting large amounts of EMG and other patient-related data, organizing such data for system optimization, and initiating an alarm in response to a suspected seizure or in, response to post-seizure motor manifestations. FIG. 9 illustrates an exemplary embodiment of such a system that may be configured to monitor a patient for seizure activity using the methods described herein. In the embodiment of FIG. 9, a seizure detection system 100 may include an acoustic sensor 108, a video camera 109, a detection unit 112, a base station 114, and an alert transceiver 116. The detection unit 112 may be configured as a portable and wearable device disposed on or near (or even attached to) any suitable muscle or muscle groups that may be subject to motor manifestations during a seizure. And, in some embodiments, the system 100 may include any of various wireless local area network technologies. For example, a detection unit 112 may communicate wirelessly to the internet using WiFi, Bluetooth, or through another local network. And, using a local network a detection unit 112 may, in some embodiments, send data over the internet directly or via an intermediate base station 114. In some embodiments, a caregiver may be contacted directly through a local network such as WiFi. A base station 114 may be connected to the internet wirelessly (such as through a local network), or may be linked to the interact through a hard connection. The detection unit 112 may comprise one or more EMG electrodes capable of detecting electrical signals from muscles at or near the skin surface of a patient and delivering those electrical EMG signals to a processor for processing. The EMG electrodes may be coupled or attached to a patient, and may, in some embodiments, be implanted within the tissue of a patient near a muscle that may be activated during a seizure. Implanted devices may, for example, be particularly amenable for some patients where EMG signals may typically be weak, such as patients with significant adipose tissue.

The base station 114 may comprise a computer capable of receiving and processing EMG signals from the detection unit 112, acoustic data from the acoustic sensor 108, and/or data from other sensors, determining from the processed signals whether a seizure may have occurred, and sending an alert to a caregiver. The alert transceiver 116 may be carried by, or placed near, a caregiver to receive and relay alerts transmitted by the base station 114 or to the internet. Other components that may be included in the system 100, including for example, wireless device 117, 118, storage database 119, electronic devices for detecting changes in the integrity of an electrode skin interface, and one or more environmental transceivers are also described in Applicant's U.S. Pat. No. 8,983,591 and other references incorporated herein.

In using the apparatus of FIG. 9, for example, a patient 120 susceptible to epileptic seizures may, for example, be resting in bed, or may be at some other location as daily living may include, and may have a detection unit 112 in physical contact with or in proximity to his or her body. The detection unit 112 may be a wireless device so that a patient may be able to get up and walk around without having to be tethered to an immobile power source or to a bulkier base station 114. For example, the detection unit 112 may be woven into a shirt sleeve, may be mounted to an armband or bracelet, or may be an implanted device. In other embodiments, one or more detection units 112 or other sensors may be placed or built into a bed, a chair, an infant car seat, or other suitable clothing, furniture, equipment and accessories used by those susceptible to seizures. The detection unit 112 may comprise a simple sensor, such as an electrode, that may send signals to the base station 114 for processing and analysis, or may comprise a “smart” sensor having some data processing and storage capability. A detection unit 112 may include One or more smart client applications. In some embodiments, a simple sensor may be connected via wire or wirelessly to a battery-operated transceiver mounted on a belt worn by the person. In some embodiments, a detection unit 112 may be configured with a pattern database and/or include a processor configured to execute instructions for construction of one or more subset groups of peaks from an initial peak set.

The system 100 may monitor the patient 120, for example, while resting, such as during the evening and nighttime hours. If the detection unit 112 on the patient detects a seizure, the detection unit 112 may communicate via wire or wirelessly, e.g., via a communications network or wireless link, with the base station 114, to a remote cell phone 117 or other hand held or desktop device 118 via Bluetooth or simultaneously to a base station and remote cell phone 117 or other device 118. In some embodiments, a detection unit 112 may send some signals to the base station device for more thorough analysis. For example, in some embodiments, a base station 114 may include amore extensive database of patterns suitable for comparison, to one or more portions of EMG data.

In some embodiments, the detection unit 112 may process and use EMG signals (and optionally, or in some embodiments, ECG, temperature, orientation sensors, saturated oxygen, and/or audio sensor signals) to make an initial assessment regarding the likelihood of occurrence of a seizure, and may send those signals and its assessment to the base station 114 for separate processing and confirmation. If the base station 114 confirms that a seizure is likely occurring, then the base station 114 may initiate an alarm for transmission over the network 115 to alert a designated individual by way of email, text, phone call, or any suitable wired or wireless messaging indicator. It should be appreciated that the detection unit 112 may, in some embodiments, be smaller and more compact than the base station 114 and it may be convenient to use a power supply with only limited strength. Therefore, it may be advantageous, in some embodiments, to control the amount of data that is transferred between the detection unit 112 and the base station 114 as this may increase the lifetime of any power supply elements integrated in the detection unit 112. In some embodiments, if one or more of the detection unit 112, the base station 114, or a caregiver, e.g., a remotely located caregiver monitoring signals provided from the base station, determines that a seizure may be occurring, a video monitor 109 may be triggered to collect video information of the patient 120.

The base station 114, which may be powered by a typical household power supply and contain a battery for backup, may have more processing, transmission and analysis power available for its operation than the detection unit 112, may be able to store a greater quantity of signal history, and evaluate a received signal against that greater amount of data. The base station 114 may communicate with an alert transceiver 116 located remotely from the base station 114, such as in the bedroom of a family member, or to a wireless device 117, 118 carried by a caregiver or located at a work office or clinic. The base station 114 and/or transceiver 116 may send alerts or messages to designated people via any suitable means, such as through a network 115 to a cell phone 117, PDA 118 or other client device. The system 100 may thus provide an accurate log of seizures, which may allow a patient's physician to understand more quickly the success or failure of a treatment regimen. Of course, the base station 114 may simply comprise a computer having installed a program capable of receiving, processing and analyzing signals as described herein, and capable of transmitting an alert. A base station 114 may include one or more smart client applications. In other embodiments, the system 100 may simply comprise, for example, EMG electrodes as part of a device configured to transmit signal data to a smartphone, such as an iPhone, configured to receive EMG signals from the electrodes for processing the EMG signals as described herein using an installed program application. In further embodiments, so-called “cloud” computing and storage may be used via network 115 for storing and processing the EMG signals and related data. In yet other embodiments, one or more EMG electrodes may be packaged together as a single unit with a processor capable of processing EMG signals as disclosed herein and sending an alert over a network. In other words, the apparatus may comprise a single item of manufacture that may be placed on, a patient and that does not require a base station or separate transceiver. Or the base station may be a smartphone or tablet, for example.

In the embodiment of FIG. 9, the EMG signal data may be sent to a remote database 119 for storage. In some embodiments, EMG signal data may be sent from a plurality of patients with epilepsy to a central database 119 and “anonymized” to provide a basis for establishing and refining generalized “baseline” sensitivity levels and signal characteristics of an epileptic seizure. The database 119 and base station 114 may be remotely accessed via network 115 by a remote computer 113 to allow updating of detector unit and/or base station software, and data transmission. And, in some embodiments, the remote computer 113 or another computer, may also serve to monitor exchange of data including alarm signals and EMG signal data between different devices associated with any number of designated individuals set to receive the signal. The base station 114 may generate an audible or visible alarm, as may a remote transceiver 116 or detection unit 112. All wireless links may be two-way for software, and data transmission and message delivery confirmation. The base station 114 may also employ one or all of the messaging methods listed, above for seizure notification. The base station 114 or detection unit 112 may provide an “alert cancel” button to terminate an incident warning.

In some embodiments, a transceiver may additionally be mounted within a unit of furniture or some other structure, e.g., an environmental unit or object. If a detection unit 112 is sufficiently close to that transceiver, such a transceiver may be capable of sending data to a base station. Thus, the base station 114 may be aware that information is being received from that transducer, and therefore the associated environmental unit. In some embodiments, a base station 114 may select a specific template file, e.g., such as including threshold values and other data as described further herein, that is dependent upon whether or not it is receiving a signal from a certain transceiver. Thus, for example, if the base station 114 receives information from a detector 112 and from a transducer that is associated with a bed or crib, it may treat the data differently than if the data is received from a transducer associated with another environmental unit, such as, for example, clothing typically worn while an individual may be exercising or an item close to a user's sink where for example a patient may brush his or her teeth. More generally, a monitoring system may, in some embodiments, be configured with one or more elements with global positioning (GPS) capability, and position information may be used to adjust one or more routines that may be used in a detection algorithm. For example, GPS capability may be included along with or among one or more microelectromechanical sensor elements included in a detection unit 112.

The embodiment of FIG. 9 may be configured to be minimally intrusive to use while sleeping or minimally interfere in daily activities, may require a minimum of electrodes such as one or two, may require no electrodes to the head, may detect a seizure with motor manifestations, may alert one or more local and/or remote sites of the presence of a seizure, and may be inexpensive enough for home use.

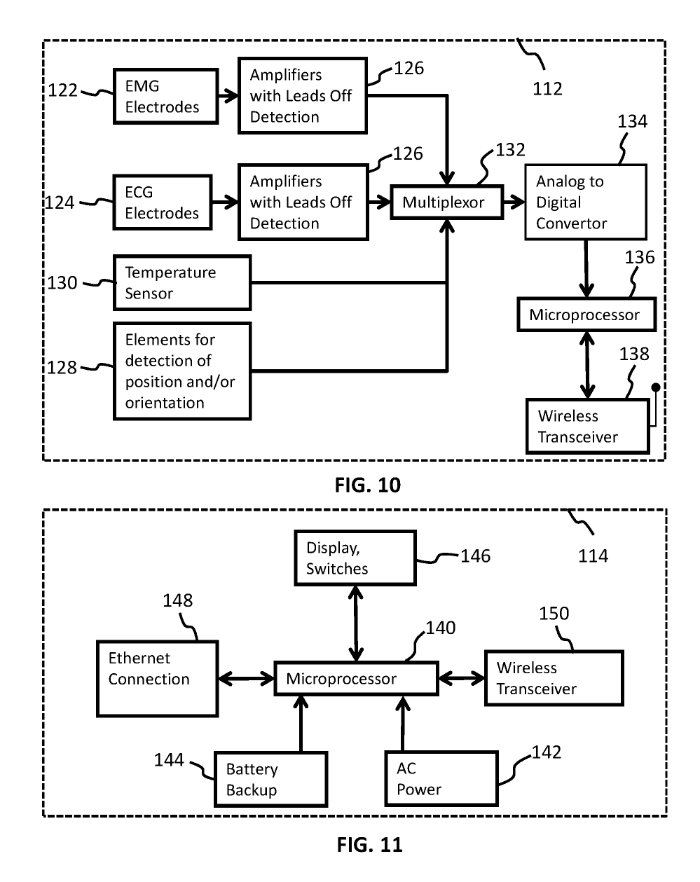

FIG. 10 illustrates an embodiment of a detection unit 112 or detector. The detection unit 112 may include EMG electrodes 122, and may also include, in some embodiments, ECG electrodes 124. The detection unit 112 may further include amplifiers with leads-off detectors 126. In some embodiments, one or more leads-off detectors may provide signals that indicate whether the electrodes are in physical contact with the person's body, or otherwise too far from the person's body to detect muscle activity, temperature, brain activity or other patient phenomena. The detection unit 112 may further include one or elements 128, such as solid state microelectromechanical (MEMS) structures, configured fur detection of position and/or orientation of the detection unit 112. For example, an element 128 may include one or more micromachined inertial sensors such as may include one or more gyroscopes, accelerometers, magnetometers or combinations thereof.

The detection unit 112 may further include a temperature sensor 130 to sense the person's temperature and one or more orientation or position sensitive elements 128. Other sensors (not shown) may be included in the detection unit, as well, such as accelerometers, microphones, and oximeters. Signals from electrodes 122 and 124, temperature sensor 130, orientation and/or position sensors 126 and other sensors may be provided to a multiplexor 132. The multiplexor 132 may be part of the detection unit 112 or may be part of the, base station 114 if the detection unit 112 is not a smart sensor. The signals may then be communicated from the multiplexor 132 to one, or more analog-to-digital converters 134. The analog-to-digital converters may be part of the detection unit 112 or may be part of the base station 114. The signals may then be communicated to one or more microprocessors 136 for processing and analysis as disclosed herein. The microprocessors 136 may be part of the detection unit 112 or may be part of the base station 114. The detection unit 112 and/or base station 114 may further include memory of suitable capacity. The microprocessor 136 may communicate signal data and other information using a transceiver 138. Communication by and among the components of the detection unit 112 and/or base station 114 may be via wired or wireless communication.

Of course, the exemplary detection unit of FIG. 10 may be differently configured. Many of the components of the detector of FIG. 10 may be in base station 114 rather than in the detection unit 112. For example, the detection unit may simply comprise an EMG electrode 122 in wireless communication with a base station 114. In such an embodiment, A-D conversion and signal processing may occur at the base station 114. If an ECG electrode 124 is included, then multiplexing may also occur at the base station 114.

In another example, the detection unit 112 of FIG. 10 may comprise an electrode portion having one or more of the EMG electrode 122, ECG electrode 124 and temperature sensor 130, in wired or wireless communication with a small belt-worn transceiver portion. The transceiver portion may include a multiplexor 132, an A-D converter 134, microprocessor 136, transceiver and other components, such as memory and I/O devices (e.g., alarm cancel buttons and visual display).

FIG. 11 illustrates an embodiment of a base station 114 that may include one or more microprocessors 140, a power source 142, a backup power source 144, one or more I/O devices 146, and various communications means, such as an Ethernet connection 148 and transceiver 150. The base station 114 may have more processing and storage capability than the detection unit 112, and may include a larger electronic display for displaying EMG signal graphs for a caregiver to review EMG signals in realtime as they are received from the detection unit 112 or historical EMG signals from memory. The base station 114 may process EMG signals and other data received from the detection unit 112. If the base station 114 determines that a seizure is likely occurring, it may send an alert to a caregiver via transceiver 150.

Various devices in the apparatus of FIGS. 9-11 may communicate with each other via wired or wireless communication. The system 100 may comprise a client-server or other architecture, and may allow communication via network 115. Of course, the system 100 may comprise more than one server and/or client. In other embodiments, the system 100 may comprise other types of network architecture, such as a peer-to-peer architecture, or any combination or hybrid thereof.

Generally, the devices of a seizure detection system may be of any suitable type and configuration to accomplish one or more of the methods and goals disclosed herein. For example, a server may comprise one or more computers or programs that respond to commands or requests from one or more other computers or programs, or clients. The client devices may comprise one or more computers or programs that issue commands or requests for service provided by one or more other computers or programs, or servers. The various devices in FIG. 9 may be servers or clients depending on their function and configuration. Servers and/or clients may variously be or reside on, for example, mainframe computers, desktop computers, PDAs, smartphones (such as Apple's iPhone™, Motorola's Atrix™ 4G, Motorola's Droid™, Samsung's Galaxy S™, Samsung's Galaxy Note™, and Research In Motion's Blackberry™ devices), tablets (such as Sony's Xperia™, Samsung's Galaxy Tab™, and Amazon Kindle™) netbooks, portable computers, portable media players with network communication capabilities (such as Microsoft's Zune HD™ and Apple's iPod Touch™ devices), cameras with network communication capabilities, smartwatches, wearable computers, and the like.

A computer may be any device capable of accepting input, processing the input according to a program, and producing output. A computer may comprise, for example, a processor, memory and network connection capability. Computers may be of a variety of classes, such as supercomputers, mainframes, workstations, microcomputers, PDAs and smartphones, according to the computer's size, speed, cost and abilities. Computers may be stationary or portable, and may be programmed for a variety of functions, such as cellular telephony, media recordation and playback, data transfer, web browsing, data processing, data query, process automation, video conferencing, artificial intelligence, and much more.

A program may comprise any sequence of instructions, such as an algorithm, whether in a form that can be executed by a computer (object code), in a form that can be read by humans (source code), or otherwise. A program may comprise or call one or more data structures and variables. A program may be embodied in hardware or software, or a combination thereof. A program may be created using any suitable programming language, such as C, C++, Java, Perl, PHP, Ruby, SQL, and others. Computer software may comprise one or more programs and related data. Examples of computer software include system software (such as operating system software, device drivers and utilities), middleware (such as web servers, data access software and enterprise messaging software), application software (such as databases, video games and media players), firmware (such as device specific software installed on calculators, keyboards and mobile phones), and programming tools (such as debuggers, compilers and text editors).

Memory may comprise any computer-readable medium in which information can be temporarily or permanently stored and retrieved. Examples of memory include various types of RAM and ROM, such as SRAM, DRAM, Z-RAM, flash, optical disks, magnetic tape, punch cards, EEPROM. Memory may be virtualized, and may be provided in, or across one or more devices and/or geographic locations, such as RAID technology. An I/O device may comprise any hardware that can be used to provide information to and/or receive information from a computer. Exemplary I/O devices include disk drives, keyboards, video display screens, mouse pointers, printers, card readers, scanners (such barcode, fingerprint, iris, QR code, and other types of scanners), RFID devices, tape drives, touch screens, cameras, movement sensors, network cards, storage devices, microphones, audio speakers. styli and transducers, and associated interfaces and drivers.

A network may comprise a cellular network, the Internet, intranet, local area network (LAN), wide area network (WAN), Metropolitan Area Network (MAN), other types of area networks, cable television network, satellite network, telephone network, public networks, private networks, wired or wireless networks, virtual, switched, routed, fully connected, and any combination and subnetwork thereof. The network may use a variety of network devices, such as routers, bridges, switches, hubs, repeaters, converters, receivers, proxies, firewalls, translators and the like. Network connections may be wired or wireless, and may use multiplexers, network interface cards, modems, IDSN terminal adapters, line drivers, and the like. The network may comprise any suitable topology, such as point-to-point, bus, star, tree, mesh, ring and any combination or hybrid thereof.

Wireless technology may take many forms such as person-to-person wireless, person-to stationary receiving device, person-to-a-remote alerting device using one or more of the available wireless technology such as ISM band devices, WiFi, Bluetooth, cell phone SMS, cellular (CDMA2000, WCDMA, etc.), WiMAX, WLAN, and the like.

Communication in and among computers, devices and network devices may be accomplished using a variety of protocols. Protocols may include, for example, signaling, error detection and correction, data formatting and address mapping. For example, protocols may be provided according to the seven-layer Open Systems Interconnection model (OSI model), or the TCP/IP model.

Although the disclosed method and apparatus and their advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition, or matter, means, methods and steps described in the specification. For example, any feature described for one embodiment may be used in any other embodiment. Use of the word “include,” for example, should be interpreted as the word “comprising” would be, i.e., as open-ended. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods or steps.

What is claimed is:

1. A method of monitoring a patient for abnormal motor manifestation activity patterns indicative of seizure activity comprising:
collecting an electromyography signal;
detecting a plurality of peaks included in said electromyography signal;
eliminating peak members from said plurality of peaks if said peak members fail to meet one or more first qualification thresholds;
determining a remaining group of peaks that remain following said eliminating of peak members;
constructing multiple test subgroups of peaks constructible from said remaining group of peaks; and
identifying group members among said multiple test subgroups of peaks that meet one or more aggregate qualification thresholds in order to determine one or more qualified groups of peaks.

2. The method of claim 1 wherein said one or more aggregate qualification thresholds are configured to identify qualified groups of peaks that are indicative of one more patterns of motor manifestation activity selected from a group of clonic-phase seizure activity and non-epileptic psychogenic seizure activity.

3. The method of claim 1 further comprising combining groups among said one or more qualified groups of peaks in order to determine an overall level of qualified peak activity if multiple qualified groups are identified among said one or more qualified groups of peaks.

4. The method of claim 3 wherein determining said overall level of qualified peak activity includes determining a number of qualified peaks that are a member of at least one of said one or more qualified groups of peaks.

5. The method of claim 1 wherein said multiple test subgroups of peaks include subset combinations of consecutive peaks in a time stamped list created from said remaining group of peaks.

6. The method of claim 1 wherein said multiple test subgroup are created by ordering a time stamped group of said remaining group of peaks and selecting consecutive groups including some preselected number of peaks in said time stamped group.

7. The method of claim 1 wherein said multiple test subgroups of peaks include groups of peaks of greater than some minimum peak number.

8. The method of claim 7 wherein said minimum peak number is between about 4 to about 8 peaks.

9. The method of claim 1 wherein said multiple test subgroups of peaks include groups of non-consecutive peaks constructible from said remaining group of peaks.

10. A method of organizing electromyography data for inclusion in a searchable database of seizure data comprising:
monitoring a patient for seizure activity by collecting an electromyography signal for a monitoring time period using one or more electromyography electrodes;
processing the electromyography signal in order to identify peak data in one or more time windows of collection included in said monitoring time period;
constructing multiple test subgroups of peaks from said peak data;
identifying parts of said peak data for which a test subgroup of peaks was constructed that match one or more criteria of one more seizure patterns;
storing the peak data together with information about the one or more seizure patterns to which the peak data was matched; and
providing an interface to a caregiver configured to allow the caregiver to find the stored peak data by searching for instances of data meeting one or more of said one or more seizure patterns.

11. The method of claim 10 wherein said multiple test sub groups of peaks include subset combinations of non-consecutive peaks among said peak data.

11. An apparatus for detecting abnormal motor muscle manifestations, the apparatus comprising:
one or more electromyography electrodes configured to provide an electromyography signal representing motor muscle activity;
a processor configured to receive the electromyography signal and process the electromyography signal in order to identify peaks in one or more time windows of collection included in a monitoring time period and calculate peak property values for said peaks;
said processor configured to compare said peak property values against qualification thresholds, the qualification thresholds including at least one qualification threshold associated with a property of individual peaks and at least one aggregate qualification threshold associated with a property of a plurality of peaks;
said processor configured to qualify peaks based on whether said peaks meet said qualification thresholds; and
said processor configured to determine a level of qualified peak activity and compare said level to a threshold level of activity that if exceeded identifies the presence of abnormal motor muscle activity.

13. The apparatus of claim 12 wherein said processor is further configured to send an alarm to a caregiver if said abnormal motor muscle activity is identified.

14. The apparatus of claim 12 wherein said processor is further configured to send said peak data to a searchable database.

15. The apparatus of claim 12 wherein said processor is further configured to remove peaks that fail to meet said at least one qualification threshold associated with a property of individual peaks prior to comparing property values of a group of remaining peaks to said at least one aggregate qualification threshold.

16. The apparatus of claim 12 wherein said processor is further configured to determine a plurality of aggregate peak property values for multiple test groupings constructed from said peaks and determining whether any of said multiple test groupings meets said at least one aggregate qualification threshold.
